(19) United States
(12) Patent Application Publication
(10) Pub. No.: US 2004/0180050 A1
(43) Pub. Date: Sep. 16, 2004

(54) Inverse skewing of the lymphocyte repertoire for therapy and prevention of disease

(76) Inventor: Geoffrey W. Hoffman, Vancouver (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/808,789

(22) Filed: Mar. 23, 2004

Related U.S. Application Data
(63) Continuation of application No. 09/565,628, filed on May 4, 2000, now abandoned.
Application No. 09/565,628 is a continuation-in-part of application No. 08/251,707, filed on May 31, 1994, now abandoned.

(51) Int. Cl. A61K 39/395; C12Q 1/70

(52) U.S. Cl. 424/144.1; 435/5

(57) ABSTRACT

Methods are provided for reducing the number of HIV-infected cells or HIV infection-susceptible cells of a host. Therapeutically this is achieved by exposing the cells to a V region selective element (VRSE) which binds to T cell receptors (TCR) of a V region defined family (VRDF) that is preferentially infected with HIV. The therapeutic VRSE can be an antibody which binds to the VRDF and is capable of causing cytotoxicity of infected and infection-susceptible cells, either alone, in conjunction with host factors, or fused to a toxin. The number of HIV-infection susceptible cells can be reduced prophylactically and in some circumstances therapeutically by immunization with an antibody or T cell receptor that induces an immune response that includes antibodies that bind to the TCR of a VRDF associated with HIV infection. The latter antibodies inhibit the viability of the infected or infection-susceptible cell. Means for diagnosing and treating diseases in which there is biasing of the immune repertoire are also provided, including those circumstances where very little may be known about the causative agent or the mechanism of pathogenesis. This is accomplished by selecting monoclonal antibodies for detecting antibody repertoire changes characteristic of a disease, thereby providing a diagnosis, and perturbing the immune system repertoire in the opposite direction to the direction of skewing that is characteristic of the disease or condition, thereby providing a means of prophylaxis or therapy.

### RELATED APPLICATION

The present application is a continuation-in-part of Ser. No. 08/251,707, filed May 31, 1994, which is incorporated herein by reference.

### BACKGROUND OF THE INVENTION

Many diseases and undesired conditions are characterized by changes in the repertoire of specificities of lymphocytes and their secreted products. These include AIDS, other autoimmune diseases, cancer, allergies and immunity to organ transplants. This invention describes methods for diagnosing, preventing and reversing in whole or in part such changes in the lymphocyte repertoire.

The two main classes of lymphocytes are T cells and B cells. T cells regulate the production of antibody by B cells, and conversely B cell products (antibodies) have an impact on the T cell repertoire. Hence, if the T cell repertoire is skewed (abnormal) in a disease, the B cell repertoire is also skewed, and vice versa.

Therapeutic vaccines and anti-viral treatments have been unsuccessful to date in eliminating the infectious agent of acquired immune deficiency syndrome (AIDS), the human immunodeficiency virus (HIV), from afflicted individuals. In spite of the fact that patients often produce antibodies or cytotoxic T lymphocytes which are capable of neutralizing the virus or cells infected by the virus in vitro, such immune responses do not lead to recovery from the disease.

HIV infection typically begins with only a relatively small number of T cells (CD4⁺ lymphocytes) harboring the virus. The infection is typically accompanied by a long period of relative quiescence. During this latency period the infected person is typically asymptomatic. Gradually, however, the virus begins to emerge from its quiescence and increases in number. As the virus multiplies it undergoes a surprisingly high degree of mutation, particularly in the gene encoding the viral envelope proteins. Thus, virus which infected the patient is often different in antigenic structure from virus which is isolated at the time of clinical disease. As the disease progresses further the individual's CD4/CD8 ratio decreases, with a severe reduction in the number of CD4+ lymphocytes. Accompanying or following this decrease, the patient experiences a progressive and sometimes rapid deterioration to symptomatic AIDS.

In individuals infected with HIV, HIV-specific antibodies and/or cytotoxic T cells have been described as cross-reactive with the idiotypic determinants on CD8⁺ suppressor T cells that interact with CD4⁺ helper T cells. It is believed that idiotypic determinants on the receptors of regulatory T cells (helper T cells and suppressor T cells) are the primary targets of autoimmunity. Immunity to these idiotypes leads to the breakdown of the immune system. Hoffmann, “The Semiotics of Cellular Communication in the Immune System” (Sercarz et al., Eds.) Springer-Verlag, New York, pp. 257-271, (1988), Hoffmann et al. Proc. Natl. Acad. Sci. (USA) 88:3060-3064 (1991) and Hoffmann, Scand. J. Immunol. 41: 331-337 (1995). This idea that AIDS is an autoimmune disease suggested that an AIDS vaccine should induce tolerance rather than immunity to HIV components (see, e.g., Hoffmann et al., U.S. Pat. No. 5,230,887, incorporated herein by reference).

Although the CD4 antigen is believed to be the principal cell surface receptor for HIV, the detailed pathway by which it mediates infection is unknown. It has been proposed that the T cell receptor (TCR) acts as a coreceptor with CD4 for infection by HIV. Hoffmann et al., “New Concepts in AIDS Pathogenesis,” Montagnier and Gougeon, eds., Marcel Dekker, N.Y., pp. 273-290, (1993), and Hoffmann, Immunol. Cell Biol. 72, 338-346 (1994)). The T cell receptor repertoire is typically skewed in HIV infected individuals (Imberti et al., Science, 254, 860-862 (1991), and Rebai et al., Proc. Nat. Acad. Sci. USA, 91:1529-1533, (1994)), with some Vβ families having a stronger tendency to be infected than others. Laurence et al., Nature 358:255-259 (1992). This has been interpreted in terms of HIV being a superantigen, thus overstimulating and eventually directly killing the cell, and not in terms of the T cell receptor being involved in infecting the cell. Similarly, it has been observed that HIV-infected T cells characterized by different Vβs produce different amounts of virus in vitro. Laurence, id.

AIDS is a fatal disease for which no cure is presently known. Presently available vaccines and treatments have been unsuccessful in eliminating the infectious agent (HIV) from infected people. What is urgently needed is a new approach for decreasing virus load and possibly eliminating HIV infected cells (and ultimately also HIV) from the circulation of an HIV-infected individual. The framework for treating or preventing HIV-induced pathogenesis should also account for, among other things, the low fraction of infected helper T cells (that increases with time), the high mutation rate of HIV, the latency that precedes pathology, the shift in the quasi-species with time, the difficulty of superinfecting with a second species of HIV (or SIV in an SIV-infected animal), and in the context of the autoimmunity concept the failure of HIV recombinant protein vaccines to cause AIDS.

An extreme form of biasing of the repertoire occurs in the case of B cell lymphoma, in which case a particular B cell clone proliferates out of control. Lymphomas have been treated with anti-idiotypic antibodies that are raised against the V region of the lymphoma associated antibody. Means are needed for diagnosing and treating diseases in which there is biasing of the repertoire, without the skewing of the repertoire necessarily being characterized by a single runaway clone. In some cases very little may be known about the causative agent of a disease involving a skewed repertoire, generically referred to herein as “disease or condition X”, or “DX,” or about the mechanism of pathogenesis. Means are needed in the art for the diagnosis of such diseases, or a diagnosis of the predisposition to these diseases, together with the development of prophylactic vaccines and/or therapies for these diseases without the necessity of identifying the causative agent or the details of the pathogenic mechanism(s).

Quite surprisingly, the invention described hereinafter addresses these and other related needs by selecting monoclonal antibodies for (a) detecting antibody repertoire changes characteristic of the disease (diagnosis), and (b) perturbing the immune system repertoire in the opposite direction to the direction of skewing that is characteristic of the disease or condition (vaccine or therapy).

### SUMMARY OF THE INVENTION

In one aspect methods and compositions are described for reducing the number of HIV-infected cells of a host. HIV-infected cells or cells which are susceptible to HIV infection are exposed to an agent termed a V region selective element (VRSE), which agent binds to a T cell receptor (TCR) of a V region defined family (VRDF) associated with HIV infection (that is, the TCR of HIV-specific, HIV-infected and infection-susceptible T cells). The VRSE, acting alone or acting together with an attached toxin, inhibits the viability of the infected or infection-susceptible cell. The VRSE can be an antibody or binding fragment, including monoclonal antibodies, which binds to the VRDF and is capable of causing cytotoxicity, either alone or in conjunction with host factors as with complement or antibody-dependent cellular cytotoxicity.

The VRSE can be fused to a toxin to form a VRSE-toxin which inhibits the viability of the infected or infection-susceptible host cell. Treatment can be ex vivo, the host lymphocytes being removed from the patient and exposed to the VRSE or VRSE-toxin, then returned to the patient, or treatment can be in vivo. The treatment of the HIV-infected cells or cells susceptible to HIV infection with the VRSE or VRSE-toxin can be repeated over intervals sufficient to provide an ongoing depletion of HIV-infected cells in the host.

In addition to depleting the host lymphocyte population of cells infected by HIV or susceptible to HIV infection, non-susceptible lymphocytes can be expanded. Uninfected T cells of the host are contacted with a different VRSE such as an antibody or binding portion thereof that is not complementary to TCR of the VRDF associated with HIV infection and which is complementary to the TCRs of the population of T cells not susceptible to HIV infection. The uninfected host cells can be contacted outside of the host or the different VRSE which is not complementary to TCRs of the VRDF family which is associated with HIV infection can be administered directly to the host. The population of T cells which are not susceptible to HIV infection can be expanded or maintained at desired levels by repeating the treatment over extended intervals.

The present invention also provides compositions, including pharmaceutical compositions, which comprise a VRSE-toxin for reducing the number of HIV infected or HIV infection susceptible cells of a mammalian host. The VRSE, which may be an antibody such as a monoclonal antibody or an HIV polypeptide, binds to the TCR of a VRDF associated with HIV infection and in combination with the associated toxin is capable of depleting the population of HIV infected or infection susceptible mammalian host cells. Relevant polypeptides in this respect include gp120, gp41, p24 and Nef of HIV and fragments thereof.

In yet other aspects the invention provides vaccine compositions and methods for preventing AIDS and HIV infection by deleting HIV susceptible T cells in a host. The vaccine comprises an anti-HIV antibody or TCR, for example a TCR which specifically recognizes the V3 loop of HIV gp120, or the V3 loop-recognizing domain of said TCR, or anti-V3 loop antibodies or the V3 loop-recognizing domain of the antibodies, formulated in a form and an amount sufficient to induce an immune response which specifically inhibits the viability of the HIV infection-susceptible T cells.

More generally, the invention provides methods for diagnosing a disease or condition, or a predisposition for contracting a disease or condition, by detecting antibodies associated with the disease or condition and that are substantially absent in healthy persons. Methods are also provided for treating a disease or condition in which the lymphocyte repertoire is abnormal, by administering a substance that reacts with antibodies that are associated with the disease or condition and that are substantially absent in healthy persons. The substance used for treatment can be an antibody, an antibody coupled to a toxin., or an antibody binding fragment coupled to a toxin, and the disease or condition an autoimmune disease, cancer, allergy, etc. The disease or condition may also be prevented (autoimmune disease, cancer, allergy, or immunity to a graft) by prophylactically immunizing the individual with a substance that induces an immune response against antibodies present in individuals with the disease and substantially absent in healthy people. For example, the immunizing substance may be an antibody, an antibody binding fragment, or an agent with a shape sufficiently similar to said antibodies that they induce an immune response that reacts with the antibodies associated with the disease.

### BRIEF DESCRIPTION OF THE FIGURES

FIG. 1 illustrates core aspects of the present invention, contrasted with conventional approaches to the prevention and treatment of AIDS.. The method is known as Elimination of HIV-specific T cells (EHT), where the HIV-specific T cells are those that are infected by HIV and produce HIV. There is complementarity between HIV proteins and firstly the receptors on HIV-specific T cells, and secondly between HIV and anti-HIV antibodies. An anti-anti-HIV antibody has a shape that mimics HIV and can kill the HIV-specific T cells. Anti-anti-HIV antibodies therefore act as AIDS therapeutics, and anti-HIV antibodies function as vaccines, since injecting them can cause sustained production of anti-anti-HIV antibodies by the immune system of the immunized person that deplete HIV-specific T cells on an ongoing basis.

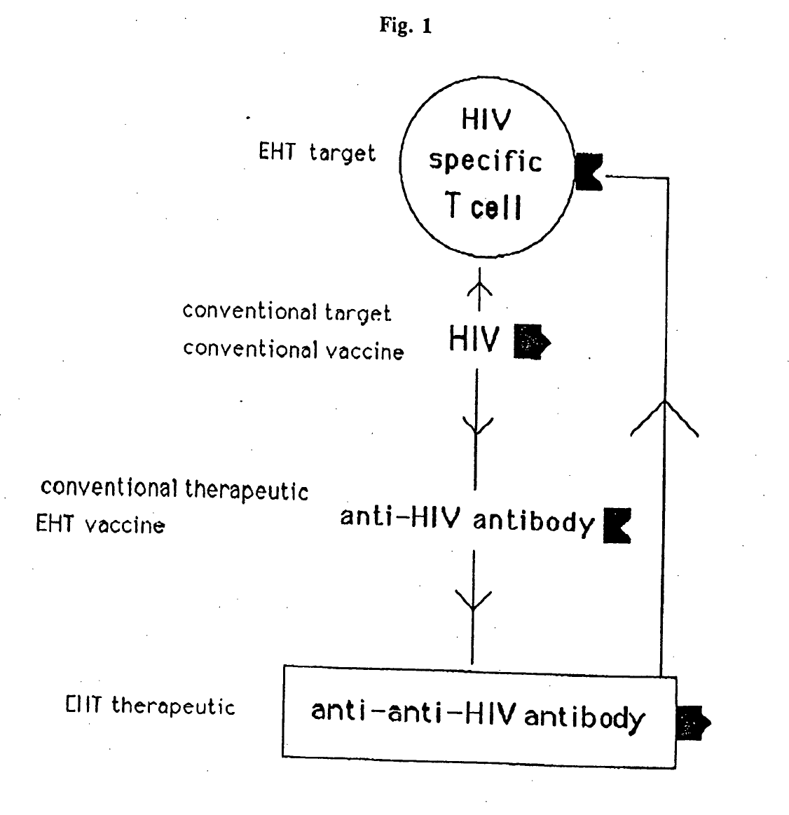

FIG. 2 shows that antibodies produced in response to immunization with a V3 loop-specific monoclonal antibody (F58) reacted with anti-HIV monoclonal antibodies MB29 and MB30 but not with F58 or with control IgM and IgG1 antibodies.

FIG. 3 shows the reactivity in ELISA of serum from ten HIV negative persons to the anti-V3 loop murine IgG1 monoclonal antibody F58 (FIG. 3A), reactivity to a control monoclonal IgG1 antibody, anti-HCG (FIG. 3B), and reactivity against F58 less the reactivity against the control antibody (FIG. 3C).

### DESCRIPTION OF THE SPECIFIC EMBODIMENTS

The present invention provides methods and compositions for decreasing HIV load in an infected individual and eliminating HIV infected cells (and ultimately also HIV). from the circulation of the HIV-infected individual. The invention also provides a means for the prevention of infection.

The methods and compositions for treating and also preventing AIDS according to the present invention are based on the preferential infection of T cells that have specificity for the virus and which express two distinguishing markers on their cell surface, namely T cell receptor and viral components such as gp120. These two markers are used together with antibodies or toxin-coupled antigen to eliminate the infected or infectable cells. The T cell receptor directed agents include for example anti-Vα, anti-Vβ or anti-idiotype antibodies, or toxin coupled HIV or HIV components. One method for the deletion of a particular V region defined family is the use of a cytotoxic form and dose of a corresponding monoclonal antibody. For example, to delete Vβ12 clones use cytotoxic anti-Vβ12 monoclonal antibodies. To more globally delete HIV infected, gp120 expressing T cells use a high affinity xenogeneic (for example murine), monoclonal anti-gp120 antibody. In addition to the elimination of HIV-specific T cells, T cells belonging to V region defined families that are not HIV-specific may be expanded using an appropriate antigenic stimulus specific for that V region defined family. If necessary, repeated applications of the process of selective depletion of infected or infectable V region defined families and selective expansion of uninfected V region defined families is used to systematically reduce the fraction of T cells that are HIV infected. While the extended TCR-autoimmunity theory of Hoffmann et al., (1993) supra, and Hoffmann, Immunol. Cell Biol. 72: 338-346 (1994), explains how HIV by itself “skews” the repertoire in one direction, leading to immunity against key idiotypic determinants of suppressor T cells and thus to disruptive autoimmunity, the present invention describes a countervailing strategy designed to skew the repertoire in the opposite direction. Selectively decreasing the number of cells that are actual or potential hosts for HIV in an infected person automatically reduces the viral load.

A vaccine for the prevention of HIV infection is also provided by the methods and compositions of the present invention. A representative sample of HIV infected individuals is surveyed to determine the V region defined families that are most frequently infected at the population level, and which ones are not infected or only rarely infected. A vaccine comprises a composition administered to specifically reduce the number of cells most frequently infected, optionally together with a composition chosen for its ability to specifically increase the number of cells which are not infected or only rarely infected.

The preferential infection of HIV-specific T cells leads to an explanation of AIDS pathogenesis that involves a remarkable interplay between the helper T cell repertoire and the repertoire of HIV variants. Offered by way of explanation and not limitation, it is believed that HIV virions are selected on the basis of having complementarity to as many helper T cell receptors as possible. A process known as coselection ensues involving the repertoire of helper T cells and the many HIV variants. Coselection is a process involving two diverse populations with mutual positive selection of some members of each of the two populations, such that selection of any member of one of the populations is dependent on interaction with (recognition of) one or more members of the other population (Hoffmann et al., 1993, supra; Hoffmann, 1994 supra, and co-pending commonly owned application U.S. Ser. No. 08/200,869, which is incorporated herein by reference). In an idiotypic network theory, there is also coselection of helper T cells and suppressor T cells (Hoffmann, 1994, supra), with the suppressor T cells being selected on the basis of being able to recognize as many helper T cell idiotypes as possible. Hence the repertoire of HIV variants and the repertoire of suppressor T cells are subject to the same selection pressure. (Hoffmann et al., The Semiotics of Cellular Communication in the Immune System, Sercarz et al., Eds., Springer-Verlag, N.Y., pp. 257-271 (1988)). Thus some of the antigenic determinants on HIV and some of the idiotypic determinants on suppressor T cells can be expected to undergo convergent selection, such that with time they resemble each other more and more from the point of view of the helper T cell repertoire. Immunity against the antigenic determinants on HIV then becomes immunity also against the idiotypic determinants on suppressor T cells. Immunity against suppressor cells means the loss of the normal regulation of helper T cells, and an unregulated helper T cell population is tantamount to autoimmunity.

A requirement for the T cell receptor to be a coreceptor or effective coreceptor for infection explains why only a minority of T cells are infected with HIV; only a minority have T cell receptors (TCR) specific for the virus. The selection process explains why the fraction of infected T cells gradually increases with time. The virus acts as an antigen, and causes the positive selection of HIV-specific T cells. At the same time, those virus particles that are most efficiently recognized by T cells are preferentially selected. Consequently there is an increase with time in the fractions of infected T cells, and of infective virus particles. The high level of HIV observed in the lymph nodes is believed to result from the most active immune response occurring there, with mutual positive selection of HIV specific helper T cells and complementary HIV variants.

This mechanism of HIV pathogenesis provides an explanation for the long latency period in HIV infection prior to the onset of AIDS. Again, while offered by way of explanation and not limitation, the latency period is believed to correspond to the time needed for the HIV quasispecies to become sufficiently similar to the endogenous suppressor T cell idiotypes such that HIV-specific immunity crossreacts with suppressor T cell idibtypic determinants and thus disrupts the normal regulation of helper cells by suppressor cells.

HIV is characterized by a high mutation rate. The conventional viewpoint is that mutations in the HIV genome permit the virus to escape the range of host immunity. The present invention is related to an alternative possibility, namely, that the high rate of mutation may facilitate the generation of HIV variants that are positively selected to recognize a progressively increasing fraction of helper T cell idiotypes. Variants with a high rate of adaptation in this sense are selected over those with a low mutation rate.

This pathogenic mechanism on which the present invention is based at least in part adds a new insidious dimension to HIV, in the sense of a seeming inevitability in the pathogenic process. It also explains why recombinant HIV protein vaccines have not caused disease when injected into uninfected people, which is paradoxical in the context of the original, more static TCR-autoimmunity model. Hoffmann, 1988, supra, and Hoffmann et al. Proc. Nat. Acad. Sci. (USA), 88:3060-3064 (1991), both of which are incorporated herein by reference. The immune response to an antigen that has a constant shape would not cause the dynamical convergence of immunity towards suppressor T cell idiotypes that can be caused by mutations and selection of the live virus. Hoffmann et al., “New Concepts in AIDS Pathogenesis”, L. Montagnier and M.-L. Gougeon, Eds., Marcel Dekker, N.Y., pp. 273-290, (1993), and Hoffmann, Immunol. Cell Biol. 72: 338-346 (1994), both of which are incorporated herein by reference.

The pathogenesis mechanism of HIV disease herein described also explains the seemingly paradoxical resistance of SIV-infected monkeys to superinfection with a second strain of SIV Daniel et al., Science 258: 1938-1941 (1992), and Stott et al., Rivista Bimestr. Biotecnol. 9: 57 (1994), and the resistance of an HIV infected cell culture to infection with a second strain or quasispecies of HIV. Offered by way of explanation and not limitation, it is believed that the first infection results in an elevated level of both a particular SIV or HIV quasispecies together with an elevated level of specifically matched CD4 T cells (and/or an elevated level of CD8 T cells that are positively selected because they are idiotypically connected to those same CD4 cells). The selected populations of T cells are an environment for the virus that is customized to suit the particular strain of SIV or HIV. A second strain or quasispecies does not have the same advantage of an environment that is idiotypically selected to suit itself, and hence cannot effectively compete against the first strain or quasispecies.

The T cell receptor repertoire is typically skewed in HIV infected individuals, with some Vβ families having a stronger tendency to be infected than others. Laurence et al., supra. Similarly, the finding that T cells characterized by different Vβs produce different amounts of virus in vitro Laurence et al., Nature 358: 255-259 (1992), incorporated herein by reference. Both of these findings were interpreted in terms of HIV being a superantigen, rather than in terms of the T cell receptor being involved in the infection step.

An understanding of the present invention may be facilitated by the introduction of two acronyms, namely VRDF for “V region defined family” and VRSE for “V region selective element.” The term VRSE is a generalization of the term Vβse (Vβ selective element) introduced by Janeway, Nature 349:459 (1991). Each VRSE is a substance that has complementarity to the T cell receptors of cells of the corresponding VRDF. For example, an anti-Vβ12 monoclonal antibody is a VRSE that has complementarity to the VRDF consisting of T cells that express Vβ12. An anti-idiotype is a VRSE that has complementarity to a VRDF consisting of a set of cells that express the corresponding idiotype (which may involve T cell receptors with quite diverse V regions genes). VRSEs useful in the present invention include, but are not limited to, (1) anti-Vα antibodies, anti-Vβ antibodies and anti-idiotypic antibodies; (2) Fab fragments, F(ab)₂ fragments and peptides derived from the preceding antibodies; (3) superantigens (Immunol. Rev. 131 (1993)), and (4) HIV virions and HIV components including glycoproteins, proteins and peptides.

There is a greater degree of cross-reactivity in the recognition of HIV-specific idiotypes by anti-idiotypes than in the recognition of the HIV variants by anti-HIV antibodies. For example, neutralizing antibodies tend to be highly strain specific, while the monoclonal antibody 1F7, for example, reacts with a variety of different anti-HIV antibodies that are found in HIV-infected people. It reacts with anti-HIV antibodies from about 70% of infected people (Muller et al., J. Immunol., 147, 933-941, 1991; Wang et al., Eur. J. Immunol., 22, 1749-1755, 1992). 1F7 also blocks killing of CD4 cells by killer T cells obtained from HIV-specific T cells, presumably by blocking the T cell receptor of the killer T cells (Grant, U.S. patent application Ser. No. 08/241,897, incorporated herein by reference). It even reacts with anti-SIV antibodies made by rhesus macaque monkeys (Grant et al., J. Leuk. Biol., 44:545, 1993). Another anti-anti-HIV monoclonal antibody, namely YT3, also has broad reactivity; it binds to antibodies that are present in approximately 35% of HIV-infected persons. Taken together, these findings indicate that immunization with an anti-HIV antibody can induce anti-anti-HIV antibodies that effectively decrease the level of infectivity of CD4 T cells. In other words, anti-anti-HIV antibodies are likely to be an effective VRSE for targeting the VRDF defined by HIV-specificity and susceptibility to HIV infection.

A rationale for a high level of cross-reactivity in the idiotypes expressed on HIV-specific T cells, in spite of a high level of variability in HIV itself, emerges from the extended TCR-autoimmunity model of AIDS pathogenesis (Hoffmann et al., “New Concepts in AIDS Pathogenesis”, L. Montagnier and M.-L. Gougeon, Eds., Marcel Dekker, N.Y., pp. 273-290, (1993),. and Hoffmann, Immunology and Cell Biology, 72, 338-346, 1994, Hoffmann, Scand. J. Immunol., 41, 331-337, 1995). In this model, helper T cells responding to HIV are subject to three selective constraints, namely the recognition of MHC-II, recognition of suppressor T cell idiotypes, and recognition of HIV. Such constrained selection is likely to result in the selection of idiotypes that are more narrowly defined and hence more cross-reactive than would be the case were they selected solely on the basis of recognition of HIV. The constrained selection of helper T cell idiotypes impacts also on the selection of B cells, and may indirectly explain the high level of cross-reactivity of anti-idiotypes against anti-HIV antibodies that is described herein.

Peripheral blood lymphocytes are obtained from an HIV infected individual and different VRDFs are analyzed for their relative levels of infection. For example, the lymphocytes are analyzed on the basis of their Vβ families, without precluding the possibility that some other VRDF marker can be used in the same way in some cases. The level of infection of various Vβ families is determined. This can be achieved by cell sorting using anti-Vβ family antibodies together with quantitative PCR (polymerase chain reaction) for HIV to determine the level of infection of each sorted Vβ family sample. Alternatively, the same information can be obtained by flow cytometry with double staining of T cells using anti-Vβ family and anti-gp120 antibodies. Laurence et al. Nature, 358: 255-259 (1992).

Therapy according to the present invention thus involves skewing the repertoire back towards the normal repertoire in order to reverse the progressive increase in. infection of CD4 cells. This can be achieved using VRSEs that distinguish between T cells that are infected and T cells that are not infected.

The methods and compositions of the present invention involve using either the T cell receptor or HIV proteins as the marker of infectable and infected cell targets. In the case of the T cell receptor being the target, the following methods may be applied: (1) use one or more VRSEs to delete the cells that are preferentially infected by the virus, or (2) use other VRSEs to expand the populations that are not infected, or (3) combine (1) and (2). These methods change the helper T cell repertoire environment of the virus, such that with time a smaller and smaller fraction of T cells are susceptible targets for virus infection and replication. The systematic change in the virus environment that is engineered in this way causes an exponential decrease in the viral load. Eventually the virus may even be eliminated from the cells and circulation of the infected individual.

When the HIV infected cells have been classified (assigned to a particular VRDF) they are deleted using corresponding cytotoxic V region selective elements (VRSEs), for example antibodies specific for a Vβ family. The antibody may be inherently cytotoxic, or may be coupled to a toxin. A variety of cytotoxic molecules are suitable for use as the cytotoxic domain in the toxin conjugates described here. Any toxin known to be useful as the toxic component of an immunotoxin may be used, and preferably a protein toxin that may be recombinantly expressed. Particularly useful as the cytotoxic domain are bacterial toxins such as Pseudomonas exotoxin A (PE), diphtheria toxin, shiga toxin and shiga-like toxin, and ribosome inactivating toxins derived from plants and fungi, including ricin, α-sarcin, restrictotocin, mitogellin, tricanthosin, saporin-G, saporin-1, momordin, gelonin, pokeweed antiviral protein, abrin, modeccin and others described in Genetically Engineered Toxins, ed. A. Frankel, Marcel Dekker, Inc. (1992), incorporated by reference herein; and any recombinant derivatives of those proteins. See generally, Olsnes and Pihl, Pharmac. Ther. 25:355-381 (1982) and U.S. Pat. Nos. 4,675,382 and 4,894,443 which describe fusion proteins containing diphtheria toxin fragments, each incorporated by reference herein. Also useful as cytotoxic agents coupled to or otherwise targeted by the monoclonal antibodies or fragments thereof are mammalian derived (preferably human) proteins with ribonucleolytic activity, such as ribonucleases engineered to be potent cytotoxins.

The toxin molecules may be fused to, or otherwise bound to a monoclonal antibody or binding fragment thereof or an HIV protein used as the targeting component by methods generally known and available to those skilled in the art. The two components may be chemically bonded together by any of a variety of well-known chemical procedures. For example, the linkage may be by way of heterobifunctional cross-linkers, e.g. SPDP, carbodiimide, glutaraldehyde, or the like. The toxin molecules may also be fused to the antibody or binding regions or HIV proteins by recombinant means, such as through the production of single chain antibodies. The genes encoding protein chains may be cloned in cDNA or in genomic form by any cloning procedure known to those skilled in the art. See for example Sambrook et al., Molecular Cloning: A Laboratory Manual, Cold Spring Harbor Laboratory Press, Cold Spring Harbor, N.Y. (1989). The recombinant production of various immunotoxins is well-known within the art and can be found, for example in Thorpe et al., Monoclonal Antibodies in Clinical Medicine, pp. 168-190 Academic Press, N.Y. (1982), Waldmann, Science, 252:1657 (1991), and Pastan et al., Ann. Rev. Biochem. 61:331-354 (1992)) each of which is incorporated herein by reference.

Effective dose levels for cytotoxic antibodies can be determined by general methods known in the field, and by taking into account other considerations such as the amounts of antibodies that have been used to modulate the level of expression of idiotypes, allotypes, isotypes, Vα families and Vβ families in animal models. A dose of the order of from about 20 mg up to about 200 mg or more for a 70 Kg person may be administered, typically parenterally, more preferably intravenously, based in part on results obtained with 10-100 μg of anti-Vβ antibodies or of anti-idiotypic antibodies (idiotype binding capacity) in animal studies (Acha-Orbea et al., Cell 54:263-273, and Eichmann et al., Eur. J. Immunol. 5: 661-666 (1975), incorporated herein by reference.

T cells belonging to VRDFs that have no detectable infection can be expanded by the administration of substances specific for the VRDFs in a form that is known to selectively stimulate expansion of the clones. They may be administered with or separate from substances used to delete the HIV infected or infectable cells. The selective stimulation may be initiated, for example, by administration of a relatively low dose of antibodies that are known to be able to stimulate T cells. For example, 200 μg of IgG antibodies given intravenously is generally sufficient for a 70 Kg person, based in part on the amount of anti-idiotype that can prime the T cells of a mouse for an antigen-specific response (100 ng of idiotype binding capacity (Eichmann et al., Eur. J. Immunol. 5:661-666 (1975)), although the dosage may be adjusted considerably by one skilled in the art to accommodate a variety of clinical conditions so as to achieve the desired level of stimulation.

Another embodiment of the present invention is to use inactivated HIV or fragments thereof as the VRSE for the depletion part of the inverse skewing. Toxins are coupled to the VRSE, as with the coupling of toxins to monoclonal antibodies and fragments as described herein, and the resulting conjugate kills HIV specific T cells. The CD4 binding site of gp120 is deleted or otherwise made non-functional on the VRSE, since otherwise all CD4 cells, rather than just the HIV-specific T cells are killed. The neutralization of HIV by many anti-V3loop antibodies suggests, in the context of the present invention, that the V3 loop of gp120 is a part of the virus that typically interacts with the T cell receptor, although this is offered by way of possible explanation, not limitation. An appropriate therapeutic is a viral component such as the V3 loop coupled to a toxin. V3 loops prepared from host HIV or utilizing a cocktail consisting of V3 loops from several HIV variants or a conjugate consisting of several V3 loops, in each case coupled to a toxin are compositions useful in the present invention. It is counterintuitive and surprising that this embodiment of the invention involves using attenuated HIV or HIV fragments with a toxin to delete CD4 T cells, when the conventional wisdom is that HIV mediated killing of CD4 cells is the essence of the disease. The treatment of the present invention will preferentially deplete, however, only a subset of the CD4 cells, namely the ones that would otherwise get infected by the virus and facilitate its replication. Anti-idiotypic antibodies that bind to V3 specific antibodies and which may mimic the V3 loop and therefore bind to V3 specific T cell receptors, could also be used therapeutically, with or without toxins coupled to them.

A VRSE that preferentially stimulates the VRDF consisting of HIV uninfected cells on a population wide as opposed to individual-specific basis interacts with T cell receptors that are generically different from the helper T receptors that bind HIV. The T cell receptors of CD8 T cells are selected to have an affinity for MHC-I while CD4 cells are selected to have affinity for MHC class II. HIV is selected to have affinity primarily for helper T cell receptors, which are different from CD8 TCR. Allogeneic class I MHC, being a stimulus that preferentially stimulates CD8 cells, is thus a VRSE for preferentially stimulating the expansion of non-HIV specific, non-infected T cells. Another VRSE is anti-I-J antibody. I-J is a phenomenon of murine immunology, and is a marker primarily associated with suppressor T cells. Murphy, Ann. Rev. Immunol. 5: 405-427 (1987). Anti-I-J antibodies bind not only to a small population of mouse T cells but also to a subset of human CD8 T cells (Lehner et al., J. Clin. Exp. Immunol. 58:410-419, incorporated herein by reference). Anti-I-J antibodies are believed to have anti-MHC-image specificity (Hoffmann, 1988, supra), and they may thus be used in the methods of the present invention to stimulate T cells that have receptors that are clearly different from (in fact complementary to) the set of HIV-specific helper T cell receptors.

When the HIV-infected cell is to be deleted, HIV proteins expressed on the cells can be used as the target. The patient is treated with xenogeneic cytotoxic anti-HIV monoclonal antibodies. Even though a strong anti-gp120 immune response typically occurs in HIV infected people, it is not effective in clearing the virus. This may be due to the postulated crossreaction with suppressor T cell idiotypes, such that the HIV provoked immune response is not focused on the virus, but rather on T cell idiotypes. This would explain the phenomenon of clonal dominance in HIV infection, in which early clones persist, even when the virus itself changes by mutation and selection, and is no longer recognized by the antibodies. This is supported by the fact that many of the antibodies produced in infected individuals are directed against the V3 loop of gp120, a highly variable part of the virus. HIV does not cause AIDS in species other than humans, and this may be due to any cross-reactivity between gp120 and other species' suppressor T cells being absent or less pronounced than that between gp120 and human suppressor T cell idiotypes. Hence, antibodies produced against gp120 in a hyperimmune animal such as a mouse may be more specifically directed against the antigen (rather than the T cell idiotypes) and thus in many cases may have a higher affinity for the antigen. Certain xenogeneic antibodies, given passively, are therefore expected to be more effective as therapeutics in humans for deleting infected cells than the endogenous antibodies produced by the infected individuals.

Following treatment with the selected cytotoxic or stimulatory VRSEs, or with xenogeneic anti-HIV antibodies, the patient is monitored for changes in the population levels and the levels of infection of the chosen VRDFs or overall infection level. Effective therapy produces a decline in the fraction of infected cells and an increase in the fraction of uninfected cells. The procedure is repeated over a period from weeks to months or even years as necessary, until the fraction of infected cells is substantially reduced to the point that the patient's symptoms subside and, most desirably, to the point that the person becomes PCR negative for HIV. The VRDFs targeted in successive rounds will not necessarily be the same ones since, for example, depletion of one VRDF may result in another VRDF becoming the dominant infected population. Mutations in HIV could also result in one or more initially uninfected VRDFs becoming infected with HIV.

Macrophages are also infected by HIV. There is evidence that macrophages express a receptor for antigen specific T cell factors. Evans et al., J. Exp. Med. 136: 1318-1322 (1972). These factors presumably have the same V regions as the receptors on the T cells that synthesize them. Adsorbed specific T cell factors may therefore play a role in the infection of macrophages. The infection of macrophages would then also depend on the T cell repertoire in a way that is similar to that for the infection of T cells. When the T cell repertoire is depleted of cells that have specificity for endogenous HIV, the macrophages do not have specific T cell factors on their surface capable of binding HIV, and further macrophage infection does not occur. The modification of the T cell repertoire as described above thus leads to the depletion of HIV infection in macrophages.

The T cell receptor directed autoimmunity mechanism of AIDS pathogenesis described above explains a pathological process in which two shapes reach a certain level of similarity for the disease to become manifest. The required level of similarity between the (average) shape of the HIV quasispecies and the suppressor T cell idiotypes is reached when immunity to HIV cross-reacts with suppressor T cell idiotypic determinants. In the present invention, it is not necessary to reverse precisely the skewing that HIV causes, only to inhibit the convergence of these two sets of shapes. This is a less stringent requirement than causing the repertoire to revert to its original form and is more attainable since it is believed that there are only a small number of ways that two different shapes can become similar, but a very large number of ways for them to remain different.

The targets of the methods and compositions of the present invention are the infected T cell or T cell susceptible to HIV infection, rather than the virus itself. Targeting the place of production of the virus eventually leads to the elimination of virus. Therapeutics that target the virus directly (for example, therapeutic vaccines) are typically unable to eliminate the provirus that is present in many cells.

The imposed selection pressure applied to the immune system of the infected individual may not only inhibit but may also reverse the pathogenic process. Instead of HIV and the suppressor T cell idiotypes converging in shape space, they are driven apart by the methods of the invention, and the viral load systematically decreases. It is counterintuitive and surprising that the compositions and methods of the invention which target for deletion a subset of helper T cells, the cells that are depleted in AIDS, provides a basis for an effective therapy.

The present methods also provide a basis for a vaccine. The relevant VRDFs are identified at the population level (that is, in a cross-section of infected individuals, not just at the level of a single individual), including HIV susceptible and HIV resistant VRDFs. The vaccine is used to induce an immune response that causes a long-term deletion or suppression of the level of the HIV specific (infection susceptible) T cells, optionally together with expansion of clones that are HIV non-specific (infection resistant).

One method for the deletion of HIV susceptible T cells is to induce immunity to HIV-specific T cell receptors or equivalently other molecules that mimic those receptors. The ability of many anti-V3 loop antibodies to neutralize HIV infection suggests, in the context of a TCR coreceptor model and the fact that V3 does not interact with CD4, that the V3 loop typically interacts with the TCR in the process of infection. Infection can thus be inhibited by an anti-anti-V3 loop immune response induced by immunization with V3 loop specific T cell receptors or V3 loop specific antibodies.

The vaccine can also include a stimulatory component, administered simultaneously or separately, to expand that part of the T cell repertoire with receptors that lack complementarity to HIV. For example, the stimulatory component can comprise anti-I-J antibodies that preferentially interact with class II MHC image TCR on suppressor T cells, or allogeneic MHC class I molecules that preferentially stimulate CD8 T cells rather than CD4 T cells.

The T cell repertoire is skewed in other diseases (O'Neill, Cell. Immunol. 136: 54-61 (1991), and Wucherpfennig, et al., J. Exp. Med. 175: 993 (1992), incorporated herein by reference), including both autoimmune diseases and cancer, so the methods of the present invention regarding inverse skewing are more widely applicable than to just AIDS. There are important similarities between AIDS and lupus suggesting that the pathogenesis is similar in the two diseases. Kaye, Ann. Int. Med. 11: 158-167 (1989), incorporated herein by reference. For example, a mouse lupus model (MRL-1pr/1pr) develops anti-HIV antibodies (interpreted as being anti-MHC-image and thus specific for suppressor T cell idiotypes) and MHC-image. antibodies (anti-anti-MHC, interpreted as being specific for helper T cell idiotypes). Kion et al., Science 253: 1138-1140 (1991), incorporated herein by reference. Humans with lupus also make anti-HIV antibodies, even though, like the lupus mice, they have not been exposed to HIV. Since MHC-image and anti-MHC-image immunity play important roles in an idiotypic network model of AIDS pathogenesis, this underlines the similarities between AIDS and lupus pathogenesis and thus the similarities in therapies for these diseases. Various autoimmune diseases may represent different modes of collapse of the network, each one being associated with the development of immunity to identifiable VRDFs that are characteristic of the particular disease. Independent of whether viruses are involved, the methods of the present invention are applicable. In the case of diseases for which characteristic changes in VRDF(s) have been identified, the inverse skewing methods as described above for AIDS are applicable for both prevention and therapy, using VRSE(s) specific for the identified VRDF(s). In the case of diseases for which characteristic changes in VRDF(s) have not been identified, these can be found by epidemiological studies, both by surveying people with the disease and by monitoring people in families at risk for the disease for changes in their T cell repertoires. As for AIDS, inverse skewing for both prevention and therapy of diseases characterized by a skewed T cell repertoire involves reducing the size of expanded VRDF(s) and/or expanding the size of VRDF(s) that are diminished in their level of expression using the corresponding VRSE(s). In the case of a preventive vaccine, the T cell repertoire is biased in a way that prevents or inhibits a particular mode of collapse to which an individual may be susceptible.

The anti-anti-HIV therapeutic of this invention is intended primarily for administration to HIV infected people. The anti-HIV vaccine as described herein is intended primarily for individuals who are HIV negative. The vaccine may also be of benefit to HIV positive people whose immune system is functioning sufficiently well to respond effectively to such an immunization.

Methods are provided as part of the present invention for selecting suitable vaccine molecules with anti-HIV specificity and suitable therapeutics with anti-anti-HIV specificity. For example, to select anti-V3 loop antibodies for use as an AIDS vaccine, in one exemplary method mice or rabbits or primates are immunized with anti-V3 loop antibodies and anti-anti-V3 loop antibodies are induced which are able to deplete human PBL of HIV-infectable T cells. See Examples 7 and 8 herein. Confirmation of a selected anti-V3 loop monoclonal antibody's suitability as an EHT vaccine is its ability, when used as a vaccine in a chimpanzee trial and then in human trials, to induce anti-anti-V3 antibodies and result in the subject being protected from HIV infection. This in vivo protective property can be correlated with decreased infectability of the subject's cells in vitro, and the ability to kill or deplete V3-loop specific T cells in vitro.

In an exemplary method for selecting anti-anti-HIV antibodies for use as an AIDS therapeutic, the anti-anti-HIV antibodies can be selected on the basis of their ability to deplete PBL of HIV-infectable cells using, for example, complement mediated lysis or, alternatively, anti-anti-HIV antibodies coupled to magnetic beads (see Examples 8 and 9 herein).

As shown herein, immune responses in mice to either an anti-V3 loop monoclonal antibody, namely F58 (Group I) or a 16-mer cyclic peptide derived from that antibody, caused the production of antibodies that failed to react to F58 itself, but which reacted with other monoclonal antibodies with anti-HIV specificity that have been classified as Group I, and which were obtained using alloimmune mice (see, e.g., Example 5). Hence, in one aspect a vaccine is used to induce an anti-anti-HIV response that contributes to eliminating HIV-specific T cells, without necessarily inducing an immune response to the vaccine molecule itself. This aspect permits monitoring the effects of the vaccine molecules in vaccine trials.

Many people are exposed to HIV and do not become infected. This suggests that the immune systems of the general population are close to a threshold for HIV infectability with some people on one side of the threshold and others on the other side of the threshold. A relatively small perturbation of the immune system in the correct direction may be all that is needed to take people from the HIV infection-susceptible side of the threshold to the HIV infection-protected side.

In another aspect the invention provides means for diagnosing and treating diseases in which there is biasing of the repertoire, including those circumstances where very little may be known about the causative agent or the mechanism of pathogenesis. This is accomplished by selecting monoclonal antibodies for detecting antibody repertoire changes characteristic of a disease, thereby providing a diagnosis, and perturbing the immune system repertoire in the opposite direction to the direction of skewing that is characteristic of the disease or condition, thereby providing a means of prophylaxis or therapy.

Skewing of the T lymphocyte repertoire can be readily diagnosed (Imberti et al., Science 254:860-863 (1991) and Rebai et al., Proc. Natl. Acad. Sci. USA, 91:1529-1533 (1994), each incorporated herein by reference). Skewing of the repertoire of secreted antibodies associated with a particular disease can be diagnosed by using antibodies that preferentially bind to pooled IgG from individuals that have the disease, and do not bind to antibodies from healthy individuals. Antibodies that are typically present in a disease or condition X (or in individuals who may be at risk for disease X) but are typically absent in individuals who are healthy and not at risk are referred to herein as disease-associated antibodies for disease X, or “DX”. Monoclonal antibodies with anti-DX specificity can be generated by immunizing mice (that may be tolerized for response to normal human IgG) with the pooled IgG obtained from individuals with the disease, followed by a cell fusion and selection to obtain monoclonal antibodies. The resulting anti-DX monoclonal antibodies are used to both diagnose a predisposition to develop X and to treat individuals with X. The anti-DX monoclonal antibodies can also be used directly or indirectly to obtain an anti-anti-DX (DX-like) monoclonal antibody, which can be used prophylactically as a vaccine against disease or condition X.

Anti-DX monoclonal antibody can be used to purify DX from the serum of subjects with condition X. Then DX can be used to immunize mice to produce a second generation anti-DX monoclonal antibody with a higher affinity for DX, since purified DX is used as the immunogen. Purified DX (obtained by column purification using either first or second generation anti-DX) can also be used to purify the small component of polyclonal anti-DX from healthy individuals. The monoclonal anti-DX or polyclonal anti-DX obtained from pooled normal individuals can then be used to immunize mice to obtain a monoclonal antibody that is anti-anti-DX or DX-like. This DX-like antibody can then be used as a vaccine molecule to induce the production of anti-DX antibodies in an individual who may be at risk for disease X. The immune system of that individual is then biased against becoming skewed in the way that characterizes disease X, and is therefore inhibited from developing the disease.

The following examples are offered by way of illustration, not by way of limitation.

### EXAMPLE 1

### HIV Vaccine, Chimpanzee Model

Anti-V3 loop antibodies are affinity purified from the blood of an infected individual or group of individuals. Alternatively, murine or human monoclonal HIV-neutralizing anti-V3 loop antibodies are obtained. A suitable anti-V3 loop monoclonal antibody or immunogenic fragment thereof is selected (see Example 5, below). 100 μg of the antibodies or of a peptide derived from the antibody that has the required immunogenic properties (ability to induce antibodies directed against HIV-specific T-cell receptors) are injected with an adjuvant (for example alum) into a chimpanzee on day 0. Booster injections are given as necessary with or without adjuvant at 3-4 week intervals. Vaccine efficacy is determined according to whether anti-anti-V3 loop antibodies or other anti-anti-HIV antibodies are produced, and by determining that an increase in the amount of virus needed to infect the vaccinated chimpanzee's cells in vitro. Upon detecting anti-anti-HIV antibodies or a decrease in the infectability of the chimpanzee's PBL, the chimpanzee is challenged intravenously with 10 in vivo infectious doses of HIV and displays resistance to infection.

### EXAMPLE 2

### Therapeutic Use of Anti-gp120 Antibody in the Hu-SCID Model

This Example describes the use of a murine monoclonal anti-gp120 antibody as an AIDS therapeutic in the Human-PBL-SCID system. This is a system in which severe combined immunodeficient (SCID) mice are transplanted with human peripheral blood leukocytes (hu-PBL). Human PBL's are collected from Epstein-Barr virus seronegative donors and mononuclear cells are prepared by Ficoll-Hypaque density centrifugation. Mononuclear PBLs (2×10⁷) are injected intraperitoneally into C.B-17 SCID mice that have mouse immunoglobulin levels of less than 5 μg/ml. An infectious dose of HIV is injected intraperitoneally into the hu-SCID mice about 2 to 4 weeks later. This is followed immediately and at about 7 to 14 day intervals by injections of about 100 μg of an anti-gp120 monoclonal antibody selected to have a high affinity for gp120. Control hu-PBL-SCID mice are given an equal dose of irrelevant antibody. The mice are observed for decreasing viral load and reversal of CD4 depletion compared to the control group.

### EXAMPLE 3

### Anti-Vβ/Anti-Vα Therapeutic Use in the SIV Model

SIV infected monkeys are monitored for changes in their Vα and Vβ T cell receptor repertoires following infection. Flow cytometry apparatus is used to triple label cells with antibodies specific for (1) gp110 (of SIV), (2) single Vα families and (3) single Vβ families. Some of the more than 400 α/β combinations have a much higher level of infection than others. The monkeys are treated with a combination of the corresponding anti-Vα and anti-Vβ antibodies, just one of each at a time, using IgG antibodies. This gives efficient targeting of a particular Vα/Vβ target population, due to the fact that there can be a synergy in IgG antibody mediated killing (a single IgG antibody does not kill, while two IgG antibodies next to each other on a cell surface can kill the cell). The monkeys are monitored for changes in the level of infection and population levels of each of the α/β combinations, and the procedure is repeated until the viral load is substantially depressed and CD4 counts are back to normal.

### EXAMPLE 4

### V3-Loop-Toxin Therapeutic Use in the SIV Model

An engineered antibody that has V3 loops (from gp110 of SIV) built into its V region is injected into SIV infected rhesus macaque monkeys. Alternatively, V3 loop peptides coupled to a toxin are injected into the monkeys. These conjugates target and kill helper T cells that would otherwise be infected by SIV and mediate the proliferation of SIV. This treatment eliminates potential SIV host cells with the ability to replicate SIV and leads to a reduction in virus load.

### EXAMPLE 5

### Specificity of Serum Antibodies Produced by Mice Immunized with an Anti-V3 Loop Antibody (F58) or a Cyclized CDRH3-Peptide Derived From F58

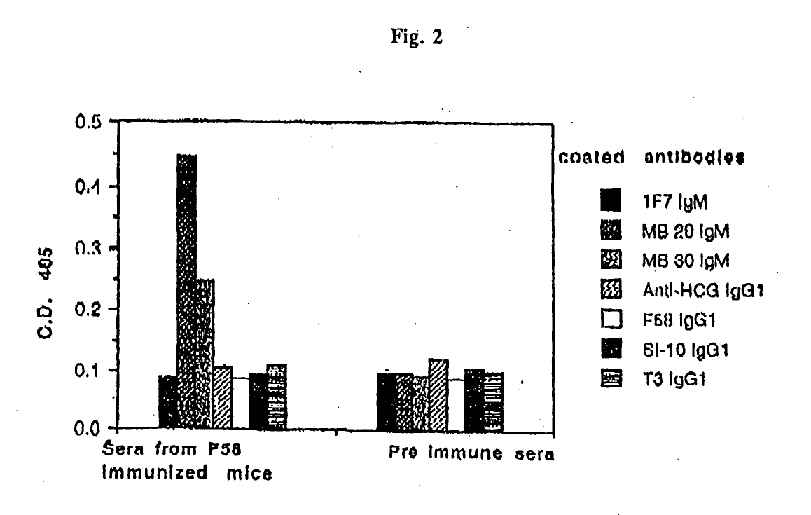

A group of four Balb/c mice were immunized once with F58, an IgG1 monoclonal antibody specific for the V3 loop of the gp120 glycoprotein of HIV together with adjuvant. The mice did not detectably produce antibodies against F58, but did produce antibodies that recognize anti-HIV monoclonal antibody, namely MB29 (FIG. 2). Similar results were obtained for binding of antibodies in the immune serum to a second monoclonal antibody called MB30. MB29 and MB30 are IgM monoclonal antibodies obtained from a fusion with cells from a C57BL/6 mouse that had been immunized with Balb/c lymphocytes. They both bind specifically in an ELISA assay to multiple HIV proteins and glycoproteins, including gp120, p24, gp41 and RT. The immune response of the Balb/c mice to F58 is an extreme example of a “heteroclitic” response, defined as a response in which the titre to the immunizing antigen is weaker than the response to a different antigen. A qualitatively identical immune response (against MB29 and MB30 but not against F58) was obtained when Balb/c mice were immunized with the same dose of a 16-mer CDRH3 cyclic peptide that has a sequence corresponding to part of the variable region of F58, and which is able to neutralize HIV (Levi et al., Proc. Nat. Acad. Sci. USA, 90:4374-4378, 1993, herewith incorporated by reference), together with the same adjuvant. This result shows that immunization with even a small, relatively easily produced peptide is capable of changing an immune system in the direction envisaged in the present invention.

Method: Mice were immunized once with 20 μg of F58 with 25 μl of TiterMax® adjuvant. Serum obtained from the mice after 83 days was assayed by ELISA for antibodies to the set of antibodies shown in FIG. 2.

ELISA assay. Immulon. III plates were coated with 1 μg/100 μl/well of antibody in carbonate buffer overnight at 4° C. The plates were emptied and blocked with 5% casein in PBS for 1 hour at room temperature. The plates were emptied and washed once with PBS-tween. Biotinylated antisera was diluted 1/500 in antibody dilution buffer and 100 μl/well was added for 1 hr at room temp. The plates were emptied and washed 3 times with PBS-tween. Avidin-alkaline phosphatase conjugate (Sigma) was diluted 1:500 and 100 μl per well was added and the plates were left for 1 hr at room temp. The plates were emptied and washed four times with PBS-tween before adding 100 μl of P-nitrophenyl phosphate substrate. The plates were incubated for 30 min. at room temp before the optical density was read at 405 nm.

As shown in FIG. 2, antibodies produced by Balb/c mice in response to immunization with a V3 loop-specific monoclonal antibody (F58) reacted with anti-HIV monoclonal antibodies MB29 and MB30 but not with F58 or with control IgM and IgG1 antibodies.

### EXAMPLE 6

### Separation of V3-Loop Binding Cells From Normal Human PBCs

Peripheral blood cells were isolated from an HIV-seronegative individual and fractionated into V3 loop-binding and non-V3 loop binding cells by incubation with biotinylated V3 peptide and avidin-coated magnetic beads. Approximately 2% of the peripheral blood lymphocytes were positively selected by this method. Precipitation with the biotinylated V3 loop peptide and avidin-coated magnetic beads resulted in three bands in a 12% SDS-polyacrylamide gel with apparent molecular masses of >200 Kd, 70 Kd and 43 Kd in independent experiments using three different donors. The molecular weight of the 43 Kd band is consistent with that of the T cell receptor. The ability to separate V3-loop binding T cells (with apparent recognition of the V3 loop by the T cell receptor) is surprising in the context of the conventional view that T cells are able to recognize antigens only together with MHC molecules. This result further validates the TCR autoimmunity (network) theory of AIDS pathogenesis and the development of the vaccines and therapeutics described herein.

### EXAMPLE 7

### Anti-Anti-V3 Loop Antibodies in Uninfected Persons

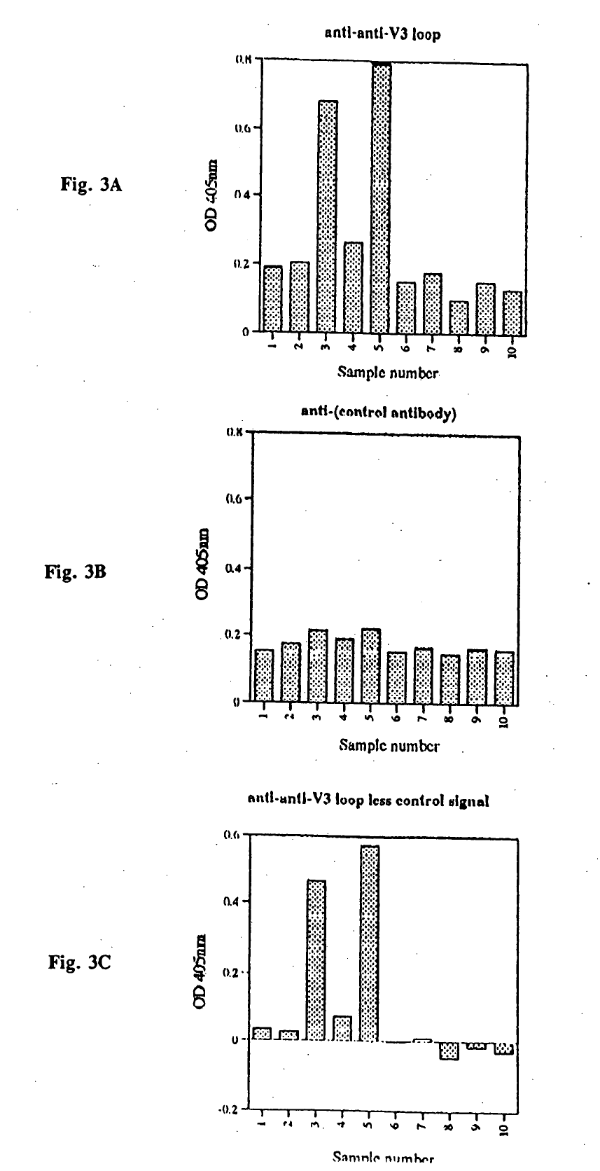

Ten people who are not infected with HIV were screened for the presence of antibodies to a murine IgG anti-V3 loop antibody, F58 (\dot{A}kerblom et al., AIDS. 4:953-960 (1990), incorporated herein by reference). Two individuals who have worked with V3 loop experimentally (but have not been immunized with anti-V3 loop) were found to be positive for anti-F58 (=anti-anti-V3 loop) antibodies (FIG. 3). This indicates that anti-anti-V3 loop antibodies can be readily made by humans.

Method: The ELISA assay was done as follows. Briefly, Immulon-III plates were coated with 100 ng/ 100 μl/well of F58 or anti-HCG in carbonate buffer overnight at 4° C. Plates were washed and 100 μl per well of human sera diluted 1 in 50 with 0.1% BSA, 1% goat serum and 5% mouse serum in antibody dilution buffer were added and incubated for 1.5 hours at room temperature. This was followed by 100 μl of goat anti-human antibody coupled to alkaline phosphatase at 1:5000 diluted in antibody dilution buffer, incubated for one hour at room temperature. Plates were washed and 100 μl/well of P-nitrophenyl phosphate added. Plates were incubated for 30 minutes and read at 405 nm.

FIG. 3 shows the reactivity in ELISA assay of serum samples from the ten HIV negative persons to the anti-V3 loop murine IgG1 monoclonal antibody F58 (FIG. 3A), reactivity to a control monoclonal IgG1 antibody, anti-HCG (FIG. 3B), and reactivity against F58 less the reactivity against the control antibody (FIG. 3C). Samples 3 and 5 have strongly elevated specific reactivity to F58.

### EXAMPLE 8

### Separation of HIV-Infectable Helper T Cells from Normal Human PBLs Using Anti-Idiotypic Antibodies

Peripheral blood lymphocytes are isolated from an HIV-seronegative individual and fractionated into (a) cells that have receptors that bind to anti-anti-HIV antibodies and (b) cells that lack such receptors by incubation with the antibody followed by goat anti-(mouse Ig)-coated magnetic beads. Enriched, depleted and unfractionated populations are then compared for infectability by HIV (Example 9).

### EXAMPLE 9

### PBL Depleted Using Anti-Anti-HIV Antibodies are Less Susceptible to HIV Infection than Unfractionated PBL

In a titration experiment, increasing amounts of a stock solution of HIV are added to (a) unfractionated PBL, (b) PBL that have been putatively depleted of HIV-specific lymphocytes using anti-anti-HIV and magnetic beads (Example 8) and (c) PBL that have been thus enriched for HIV-specific T cells. Ten samples are exposed to each concentration of virus.

Method: In brief, a virus stock is serially diluted over six logs of concentration, spanning the TCID₅₀. Aliquots of 10⁶ unfractionated PBL, or PBL depleted of anti-idiotype binding cells are exposed to dilutions of the virus stock of HIV-1 at 37° C. for 2 hours, the cells are washed and 10⁵ infected cells are dispensed to each of 10 wells of a 96 well plate. Infected cells are cultured with IL-2 for 10 days, and after the incubation cell free culture supernatant is removed and tested for the presence of HIV p24 by antigen capture ELISA (Organon Teknika, Durham, NC) or by PCR. The removal of HIV-specific T cells decreases infectability with HIV.

### EXAMPLE 10

### Monoclonal Anti-Anti-HIV Antibody as an AIDS Therapeutic

Balb/c mice were immunized with IgG pooled from HIV-infected individuals, and B-cell hybridomas were generated from the spleens of the mice. The hybridoma supernatants were screened for selective binding to IgG from HIV-infected individuals. A selected IgG₁ anti-idiotype, YT3, was used in an ELISA to assess distribution of the idiotope.

Methods: Immunization of mice and production of hybridomas. 50 μg of purified IgG pooled from HIV-infected individuals (HIVIG) (NIH AIDS reference reagent program, Rockville, Md.) in 100 μl phosphate buffered saline (PBS) emulsified with an equal volume of complete Freund's adjuvant (Sigma Chemical Company, St. Louis, Mo.). This was followed at 14 day intervals by 2 boosts with 50 μg of HIVIG in incomplete Freund's adjuvant (Sigma Chemical Company, St. Louis, Mo.). Seven days after the third injection, mice were boosted with an intravenous (i.v.) injection of 100 μg of HIVIG in 100 μl PBS via the tail vein. To produce B cell hybridomas, briefly, 3 days after i.v. boosting, the mice were sacrificed and their splenocytes isolated and fused at a 5:1 ratio with NS-1 myeloma cells in 50% polyethylene glycol (PEG) 3500 (Sigma Chemical Company, St. Louis, Mo.), followed by dilution in serum free Iscove's modified Dulbecco's medium (IMDM) (Gibco, Burlington, Ont). Following cell fusion, the mixture of cells was resuspended in petri dishes at 5×10⁶ cells/ml in semi-solid IMDM containing 20% fetal calf serum (FCS) (Gibco, Burlington, Ont), 1% hypoxanthine, aminopterin, thymidine (HAT) stock (Sigma Chemical Company, St. Louis, Mo.), 1.25 methylcellulose (Sigma Chemical Company, St. Louis, Mo.), 3×10⁶ thymocytes/ml, 2.5×10⁶ splenocytes/ml and 2.5×10⁵ NS-1 cells/ml. 7-10 days after cell fusion, growing colonies were selected and transferred to individual wells of 96 well microtiter culture plates in RPMI 1640 medium with 10% FCS and 1% penicillin/streptomycin (Gibco, Burlington, Ont. Canada).

Hybridoma screening: Hybridoma supernatants were screened for reactivity with HIVIG and non-reactivity with pooled polyclonal IgG from HIV seronegative individual (HIG) (Jackson Laboratories, Bar Harbour, Me.). Immulon III Dynatedh ELISA plates were coated with 1 μg of Goat anti-human IgG (Jackson Laboratories, Bar Harbour, Me.) overnight at 4° C. in 100 μl tris buffer (50 Mm Tris, Ph 8.2). The wells were emptied, and blocked for 60 min. with 1% bovine serum albumin (BSA) (Sigma Chemical Company, St. Louis, Mo.) in dilution buffer (50 Mm Tris, 0.8% NaCl, 0.02% NaN₃, Ph 8.0). The wells were washed once with PBS 0.5% Tween-20 (Sigma Chemical Company, St. Louis, Mo.) and 1 μg of either HIVIG or HIG in tris buffer was added to replicate wells for 90 min. Plates were washed 5 times and 100 μl of each hybridoma supernatant was transferred to individual wells for 90 min. at room temperature. Plates were again washed 5 times and bound monoclonal antibodies were detected with goat anti-mouse IgG and IgM alkaline phosphatase (AP) (Jackson Laboratories, Bar Harbour, Me.) and substrate (Sigma Chemical Company, St. Louis, Mo.). Hybridomas producing antibodies reacting selectively with HIVIG were chosen for further study, subcloned and expanded in ascites. Isotypes of selected mAb were determined with commercial isotyping kits (Pharmingen, San Diego, Calif.). Mab were purified from ascites by Protein A-sepharose (Pharmacia Fine Chemicals, Montreal, Que) or goat anti-mouse IgM-agarose (Sigma Chemical Company, St. Louis, Mo.) affinity chromatography.

Immunization of rabbit: A rabbit was immunized three times subcutaneously at 14 day intervals with 100 μg of column purified YT3. The first immunization was given with Complete Freund's Adjuvant and the second and third injections were given with Incomplete Freund's Adjuvant.

ELISA for human antibodies anti-idiotypic to YT3. Immulon 3 ELISA plates were coated overnight at 4° C. with 250 ng/well of goat anti-mouse IgG antibodies (Jackson Laboratories, Bar Harbour, Me.) in 100 μl tris buffer. The following morning the wells were emptied and blocked as above. The wells were washed once and 250 ng of either YT3 or anti-human chorionic gonadotrophin (HCG) b chain (IgG₁ isotype control) was added for 90 min. in 100 μl dilution buffer. The wells were washed 3 times and 100 μl of a 1/50 dilution of serum or plasma in dilution buffer with 10% mouse serum was added for 90 min. Wells were then washed 6 times and 100 μl of goat anti-human IgG AP (Jackson Laboratories, Bar Harbour, Me.) in dilution buffer was added/well for 60 min. Wells were washed a final 6 times and 100 μl of AP substrate added/well. Color was allowed to develop for 20 min. at room temperature and OD was read at 405 nm. The OD reading against anti-HCG was subtracted from the OD reading against YT3 and samples were considered positive if the net OD was >4 standard deviations above the mean of 209 samples obtained from blood cleared for transfusion by the Canadian Red Cross Blood Service (CRCBS).

ELISA for Serum IqG Levels: Immulon III ELISA plates were coated overnight at 4° C. with 250 ng/well of goat anti-human IgG antibodies (Caltag Hornby, Ont.) in 100 μl tris buffer. The following morning the wells were emptied and blocked for 60 min. with 200 μl of 1% BSA in dilution buffer. Serum samples were diluted 1 in 10⁶, 1 in 2×10⁶ and 1 in 4×10⁶ in dilution buffer and 100 μl incubated for 90 min. on coated ELISA plates Human IgG standards were prepared covering a range of 1 to 200 ng/ml and assayed together with unknowns. Following incubation with human standard and unknowns, plates were washed 6 times and developed as above. IgG concentrations of the test sera were estimated from the dilution yielding an OD within the linear portion of a graph of OD against IgG concentration prepared with standards of known IgG concentrations.

Serum and plasma samples: 90 plasma samples and 119 serum samples from individuals with no perceived risk for HIV infection were obtained from the CRCBS, Vancouver, Canada. These samples had been screened for hepatitis B. antigen, and for antibodies against HIV-1, HIV-2, human T-lymphotropic virus type 1 (HTLV-1) and hepatitis C virus. Serum samples from 100 western blot confirmed HIV-seropositive individuals were obtained from the British Columbia Laboratory Center for Disease Control (BCLCDC), Vancouver, Canada. Plasma samples from 166 confirmed HIV-infected individuals were obtained from the Special Immunology Services (SIS) Clinic, Chedoke-McMaster Hospitals, Hamilton, Canada.

The results showed that YT3 recognized antibodies in individual blood samples from 1.5% (3/209) of HIV-seronegative individuals with no perceived risk factors for HIV infection and 35.5% (59/166) of HIV-infected individuals. The mean level of the idiotope recognized by YT3 antibody was significantly higher (p=0.001) in the 59 YT3-reactive samples from HIV-infected individuals (0.753±0.520, mean≠S.D.) than in YT3-reactive samples from HIV-seronegative individuals (0.307±0.110). YT3 was used to column-purify YT3-reactive antibodies. These antibodies were found to react in a ELISA assay with the V3 loop of gp120 of HIV_{IIIB}. The rabbit immunized with YT3 produced antibodies that reacted with gp120 of HIV as detected by the ELISA assay.

Although the foregoing invention has been described in some detail by way of illustration and example for purposes of clarity of understanding, it will be obvious that certain changes and modifications may be practiced within the scope of the appended claims.

What is claimed is:

1. A method for reducing the number of HIV-infected cells of a host, comprising:
exposing the HIV-infected cells or cells susceptible to HIV infection to a V region selective element (VRSE) which binds to a T cell receptor (TCR) of a V region defined family (VRDF) associated with HIV infection, wherein the VRSE inhibits said infected or infection-susceptible host cell viability.

2. The method of claim 1, wherein the VRSE is an antibody or binding fragment thereof.

3. The method of claim 2, wherein the antibody is a monoclonal antibody or binding fragment thereof.

4. The method of claim 1, wherein the VRSE is fused to a toxin to form a toxin-VRSE conjugate.

5. The method of claim 1, wherein the host cells are exposed to the VRSE outside of the host.

6. The method of claim 1, wherein the host cells are contacted with the VRSE-toxin in the host.

7. The method of claim 1, further comprising the step of:
expanding a population of T cells of said host which are not susceptible to HIV infection by contacting uninfected T cells of the host with a different VRSE that is not complementary to TCR of the VRDF associated with HIV infection and which is complementary to the TCRs of said population of T cells not susceptible to HIV infection.

8. The method of claim 7, wherein the different VRSE not complementary to the TCR of the VRDF which is associated with HIV infection is an antibody or binding fragment thereof.

9. The method of claim 7, wherein the uninfected host cells are contacted outside of the host with the different VRSE not complementary to TCRs of the VRDF family which are associated with HIV infection.

10. The method of claim 7, wherein the uninfected host cells are contacted in the host with the different VRSE not complementary to TCRs of the VRDF family which are associated with HIV infection.

11. The method of claim 1, wherein the step of exposing the HIV-infected cells or cells susceptible to HIV infection with the VRSE is repeated over intervals sufficient to provide an ongoing depletion of HIV-infected cells in the host.

12. The method of claim 11, further comprising maintaining or expanding a population of T cells of said host which are not susceptible to HIV infection by repeatedly contacting T cells of the host with a different VRSE that is not complementary to TCR of the VRDF associated with HIV infection and which is complementary to the TCRs of said population of T cells not susceptible to HIV infection.

13. A composition which comprises a VRSE-toxin for reducing the number of HIV infected or HIV infection susceptible cells of a mammalian host, wherein the VRSE binds to a TCR of a VRDF associated with HIV infection, wherein the VRSE-toxin inhibits the viability of an HIV infected or infection susceptible mammalian host cell.

14. The composition of claim 13, wherein the VRSE which binds to the TCR of a VRDF associated with HIV infection is a monoclonal antibody or binding fragment thereof.

15. The composition of claim 13, wherein the VRSE which binds to the TCR of a VRDF associated with HIV infection comprises an HIV polypeptide selected from gp41, gp120, p24 or nef or a TCR binding fragment of said polypeptide.

16. The composition of claim 13, wherein the toxin which inhibits the viability of the HIV infected mammalian host cell is an inhibitor of mammalian protein synthesis.

17. The composition of claim 13, wherein the VRSE-toxin is a fusion protein.

18. The composition of claim 13, wherein the VRSE and toxin are chemically linked.

19. A method for deleting HIV-susceptible T cells in a host, comprising:
administering to the host a composition which comprises a TCR or antibody or binding fragment thereof which specifically recognizes HIV in an amount and formulation sufficient to induce an immune response which specifically inhibits the viability of said HIV susceptible cells.

20. The method of claim 19, wherein gp120 of HIV is specifically recognized by said TCR or antibody.

21. The method of claim 20, wherein the V3 loop of gp120 of HIV is specifically recognized by said TCR or antibody.

22. A method for diagnosing in a subject a disease or condition, or a predisposition for contracting a disease or condition, comprising detecting serologically in the subject antibodies associated with the disease or condition and that are substantially absent in healthy persons.

23. A method for treating a disease or condition in which the lymphocyte repertoire is abnormal, which method comprises administering a substance that reacts with antibodies that are associated with the disease or condition and that are substantially absent in healthy persons.

24. The method of claim 23, in which the substance used for treatment is an antibody, an antibody coupled to a toxin, or an antibody binding fragment coupled to a toxin.

25. The method of claim 23, in which the disease or condition is an autoimmune disease, cancer, or allergy.

26. A method for preventing a disease or condition in an individual in which the lymphocyte repertoire is abnormal, comprising immunizing the individual with a substance that induces an immune response against antibodies present in individuals with the disease and substantially absent in healthy people.

27. The method of claim 26, in which the substance used is an antibody, an antibody binding fragment, or an agent with a shape sufficiently similar to said antibodies that they induce an immune response that reacts with said antibodies.

28. The method of claim 26, in which the disease is an autoimmune disease, cancer, allergy or immunity to a graft.
